(19) United States
(12) Patent Application Publication
(10) Pub. No.: US 2022/0044804 A1
(43) Pub. Date: Feb. 10, 2022

(54) System and Method For Improved Patient Engagement And Better Data-Driven Outcomes

(71) Applicant: Brent Chase, Columbia, MD (US)

(72) Inventor: Brent Chase, Columbia, MD (US)

(21) Appl. No.: 17/396,934

(22) Filed: Aug. 9, 2021

Related U.S. Application Data
(60) Provisional application No. 63/063,539, filed on Aug. 10, 2020.

(51) Int. Cl. G16H 40/67; G16H 20/00; A61B 5/00

(52) U.S. Cl. CPC G16H 40/67; A61B 5/6802; G16H 20/00

(57) ABSTRACT

A platform system for remotely monitoring one or more behavioral event of at least one user is disclosed. The platform system includes at least one computer system having an internet connection and at least one web application, the at least one computer system having a memory for storing an array of contextual data relating to the at least one user; at least one wearable device having a processor and being communicatively connected to the at least one user and being in electronic communication with the at least one computer system, the at least one wearable device having one or more sensors for detecting physiological data of the at least one user, the at least one wearable device having a memory for storing an array of physiological data detected by the one or more sensors; wherein the processor of the at least one wearable device determines a behavioral event relating to the at least one user based on the array of physiological data in view of the array of contextual data, the processor communicating the behavioral event to the computer system; and wherein the computer system provides a notification relating to the behavioral event.

### CROSS-REFERENCE TO RELATED APPLICATIONS

This application claims the benefit of priority to U.S. Provisional Patent Application No. 63/063,539, filed on Aug. 10, 2020, which is incorporated by reference herein in its entirety.

### TECHNICAL FIELD

The present invention relates to a behavioral training tool that utilizes a modular wearable technology with integrated physiological signal sensors. More specifically, the invention relates to a remote patient monitoring system that leverages a mobile/web with wearable(s) platform for better quantification of therapeutic outcomes, increased carry-over, and increased independence of clients as well as therapists.

### BACKGROUND

One of the biggest challenges that healthcare communities face is with regards to patient carry-over or compliance. This is the completion of assigned or prescribed activities, tools, exercises, etc. that are performed either with a healthcare professional or outside (at-home, etc.) of the time with them. If one treats these interactions similar to schooling then if we only attend lectures and do no homework, the result will be the failure of a class. If one attends lectures, performs at home exercises (homework), and other activities with respect to this class then they will pass. Carry-over in healthcare functions in a similar way, if one fails to perform assigned materials or engage in similar activities outside of time with the healthcare professionals then one will not “pass” or “graduate.”

Carry-over in healthcare can be particularly problematic with children. Oftentimes parents are not involved completely or consistently with a child's therapy or treatment, as parents oftentimes are working or otherwise preoccupied with other responsibilities. Likewise, the doctors or therapists have no way of tracking parents' engagement. Bridging these divides increases parent engagement in in-home practice, helps children become more independent faster, both of which almost certainly results in better client satisfaction.

The rise of technology has led to a surge toward the quantified self. There are quantifiable physiological signals that can be collected from numerous systems of the body. Among the devices are currently on the market, few are capable of being utilized in various methods and seamlessly integrated into people's lives.

There is thus a huge opportunity to marry as well as correlate objective physiological data, client/patient engagement, and healthcare data trends to better understand (visually, numerically, etc.) independence.

### SUMMARY OF THE INVENTION

According to one non-limiting aspect of the present disclosure, an example embodiment of a physiological signal monitoring system of user(s) is disclosed. The system includes at least one computer system having an internet connection and at least one web application, the at least one computer system having a memory for storing an array of contextual data relating to the at least one user; at least one wearable device having a processor and being communicatively connected to the at least one user and being in electronic communication with the at least one computer system, the at least one wearable device having one or more sensors for detecting physiological data of the at least one user, the at least one wearable device having a memory for storing an array of physiological data detected by the one or more sensors; wherein the processor of the at least one wearable device determines a behavioral event relating to the at least one user based on the array of physiological data in view of the array of contextual data, the processor communicating the behavioral event to said computer system; and wherein the computer system provides a notification relating to the behavioral event.

According to another non-limiting aspect of the present disclosure, an example embodiment of a physiological signal monitoring system of user(s) is disclosed. The system includes at least one computer system having an internet connection and at least one web application, the at least one computer system having a memory for storing an array of contextual data relating to the patient; at least one wearable device having a processor and being communicatively connected to the patient and being in electronic communication with the at least one computer system, the at least one wearable device having one or more sensors for detecting physiological data of the patient, the at least one wearable device having a memory for storing an array of physiological data detected by the one or more sensors; wherein the processor of the at least one wearable device determines completion of a portion of said therapy exercise relating to said patient based on the array of physiological data in view of the array of contextual data, the processor communicating said completion to the computer system; and wherein the at least one wearable device provides the patient with feedback to motivate completion of the therapy exercise.

According to yet another non-limiting aspect of the present disclosure, an example embodiment of a physiological signal monitoring system of user(s) is disclosed. The system includes at least one computer system having an internet connection and at least one web application, the at least one computer system having a memory for storing an array of contextual data relating to the at least one user; at least one wearable device being communicatively connected to the at least one user and being in electronic communication with the at least one computer system, the at least one wearable device having a central processing unit operatively connected to an array of sensors for detecting physiological data of the at least one user, the at least one wearable device having a memory for storing an array of physiological data detected by the array of sensors; wherein the central processing unit of the at least one wearable device determines a behavioral event relating to the at least one user based on the array of physiological data in view of the array of contextual data, the processor communicating the behavioral event to the computer system; and wherein the central processing unit provides corrective behavior to the at least one user after detecting the behavioral event.

Additional features and advantages are described herein, and will be apparent from the following Detailed Description and the figures.

### BRIEF DESCRIPTION OF THE DRAWINGS

Features and advantages of the system and method described herein may be better understood by reference to the accompanying drawings in which:

FIG. 1 depicts a high-level flowchart demonstrating the functionality of the present invention;

FIG. 2A depicts a front view of one embodiment of an exemplar modular wearable device, as it is being worn on person's shoulder or clavicle;

FIG. 2B depicts a back view of one embodiment of an exemplar modular wearable device, as it is being worn on person's shoulder or clavicle;

FIG. 2C depicts a top perspective view of one embodiment of an exemplar modular wearable device;

FIG. 2D depicts a perspective view of the underside one embodiment of an exemplar modular wearable device;

FIG. 3A depicts another embodiment of an exemplar modular wearable device, as it is being worn on a person's upper arm;

FIG. 3B depicts another embodiment of an exemplar modular wearable device, as it is being worn on a person's forearm;

FIG. 4A depicts another embodiment of an exemplar modular wearable device, as it is being worn on a person's upper arm;

FIG. 4B depicts a perspective view of another embodiment of an exemplar modular wearable device;

FIG. 5A depicts a perspective view of another embodiment of an exemplar modular wearable device;

FIG. 5B depicts a perspective view of another embodiment of an exemplar modular wearable device;

FIG. 5C depicts a front view of another embodiment of an exemplar modular wearable device, as it is being worn across a person's chest;

FIG. 5D depicts a back view of another embodiment of an exemplar modular wearable device, as it is being worn across a person's chest;

FIG. 6 depicts a high-level flow chart demonstrating the functionality of the present invention for determination of behavioral events and subsequent actions;

FIG. 7 depicts the data flow analyzed by the present invention between the user(s), computer system, processor unit, and wearable(s) as it relates to the incorrect behavior being the desired outcome; and

FIG. 8 depicts data flow analyzed by the present invention between the user(s), computer system, processor unit, and wearable(s) as it relates to the correct behavior being the desired outcome.

A skilled artisan will appreciate the foregoing details, as well as others, upon considering the following Detailed Description of certain non-limiting embodiments of the protective gear according to the present disclosure. One of ordinary skill also may comprehend certain of such additional details upon using the protective gear described herein.

### DETAILED DESCRIPTION

The present invention is a remote patient monitoring system that leverages a platform consisting of user-inputted data mobile/web applications with physiological data wearable(s) and contextual data for better quantification of therapeutic outcomes, client/patient carry-over, and client/patient independence as well as therapists.

The present invention is a data-driven feedback closed-loop that enables data collection as well as analytics, connecting various stakeholders to better collaborate. It could serve every family in the autism community and patients with other chronic conditions. It provides correlation of the social, professional, physical, and/or emotional independence to user-inputted or client/patient carry-over to show efficiency of therapy.

The physiological data collected may include but not limited to: Photophethagrpahy (PPG), Electrocardiography (ECG), Electromyography (EMG), Heart Rate (HR), Heart Rate Variability (HRV), Respiratory Rate (RR), Blood Pressure (BP), Electrodermal Activity/Skin Conductance (EDA-perspiration), Inertial Measurement Unit (IMU), Global Positioning System (GPS). Pulse-Rate-Variability (PRV), Inter-Beat-Interval (IBI), Electrodermal Activity (EDA), Body Temperature, Peripheral Capillary Oxygen Saturation (Sp02), Peripheral Capillary Deoxygenation (SpD02), Inertial Measurement Unit (IMU), Global Positioning System (GPS), Speed (distance per time), Acceleration (distance per time squared), and/or Force (Newtons).

The contextual data collected may include but not limited to: calendar events, Rated Perceived Exertion (RPE), Emotional Perceived Exertion (EPE), dietary, exercise/exercise completion, user-inputted information, personal demographic & health information, assessments of professionals/health care professionals/care providers/therapist, environmental conditions, geographical location, altitude, temperature, humidity, and/or audio.

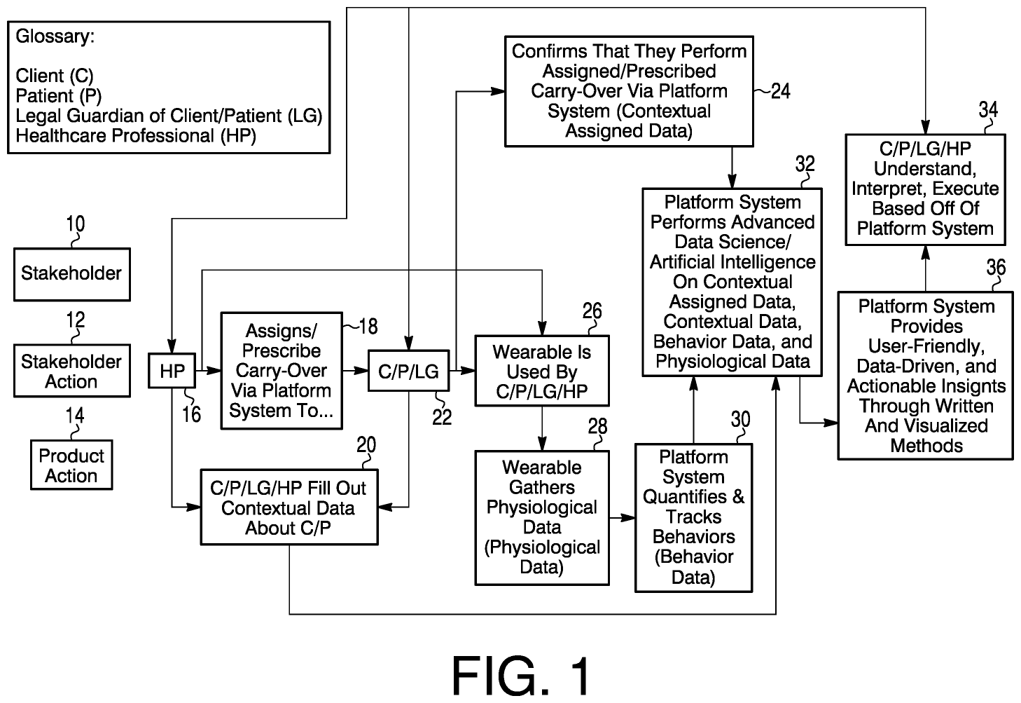

FIG. 1 depicts a high-level flowchart demonstrating the functionality of the present invention. A Healthcare Professional (HP) (reference numeral 16) assigns/prescribes carry-over therapy or treatment (reference numeral 18) via the system platform to Clients (C), Patients (P), and/or Legal Guardians of C/P (reference numerals 20, 22), any of which may be stakeholders (reference numeral 10). The C/P/LG/HP utilizes the wearable device (reference numeral 26) to gather Physiological Data (PD) (reference numeral 28) in order to have the platform system Quantify & Track (reference numeral 30) the correct and incorrect Behaviors (QTB). The C/P/LG/HP then Performs & Confirms (reference numerals 24, 32) completion or inability to complete Carry-Over (PCCO) and Contextual Data (CD) about the C/P/LG as well as the Surrounding Environment (SE). The platform then uses artificial intelligence off of the PD, QTB, PCCO, CD, and SE to provide user-friendly, data-driven, and actionable insights to the C/P/LG/HP by ways of written and/or visual methods (reference numerals 34, 36). This analysis allows C/P/LG/HP to understand, interpret, and execute more effectively based off of the platform system. In the case that the incorrect behavioral event occurs, then the system initiates the corrective behavior system, by notifying the stakeholder 10 of incorrect behavior then providing a form of haptic, visual, audio, and/or any other combination of feedback (reference numeral 12) to stakeholders. The stakeholder then undergoes the corrective behavior system action and mitigates or adjusts the incorrect behavior. In either case the system repeats the cycle of gathering physiological data and making determinations.

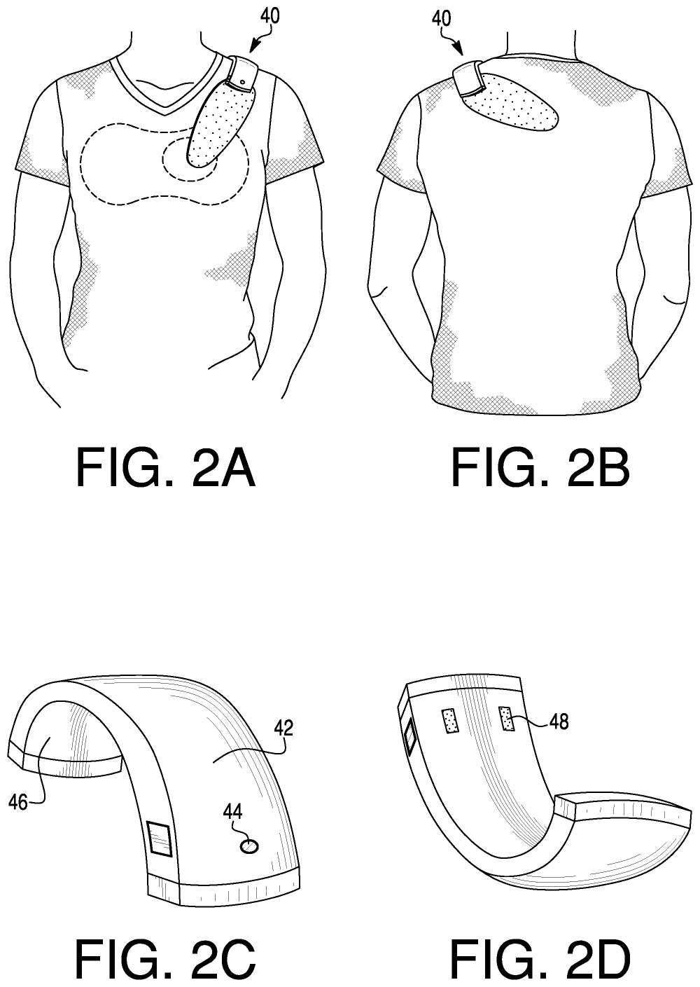

FIGS. 2A-2D depict one embodiment of an exemplar modular wearable device 40 being utilized in various methods. The wearable device 40 is curved and able to be secured in different positions on the body. As one example, the wearable device 40 is worn as an attachment to a shirt that can gather and submit physiological information. FIG. 2A shows a front view of a person wearing the wearable device 40 over the shoulder, and FIG. 2B shows the back view. The wearable device 40 has a top surface 42, which may include an LED light 44, as shown in FIG. 2C. The wearable device 40 has a bottom surface 46, which is generally contoured to fit over a shoulder and may further include ridges or ribbings, or have a rubberized coating or layer, to help maintain the placement of the wearable device 40 on the person's shirt or shoulder. The wearable device 40 may further include one or more connectors 48 for connecting the wearable device 40 to physiological sensors, such as electrocardiogram sensors (ECG, EKG, or RR sensors), as shown in FIG. 2A, for monitoring heartbeat activity, and/or electromyography sensors (EMG), as shown in FIG. 2B, for monitoring muscle response. The wearable device 40 is preferably battery operated with rechargeable batteries that may be charged on any typical charging pad or plugged into household electricity via a charging cord, such as with a USB cable or similar, as with any conventional mobile device.

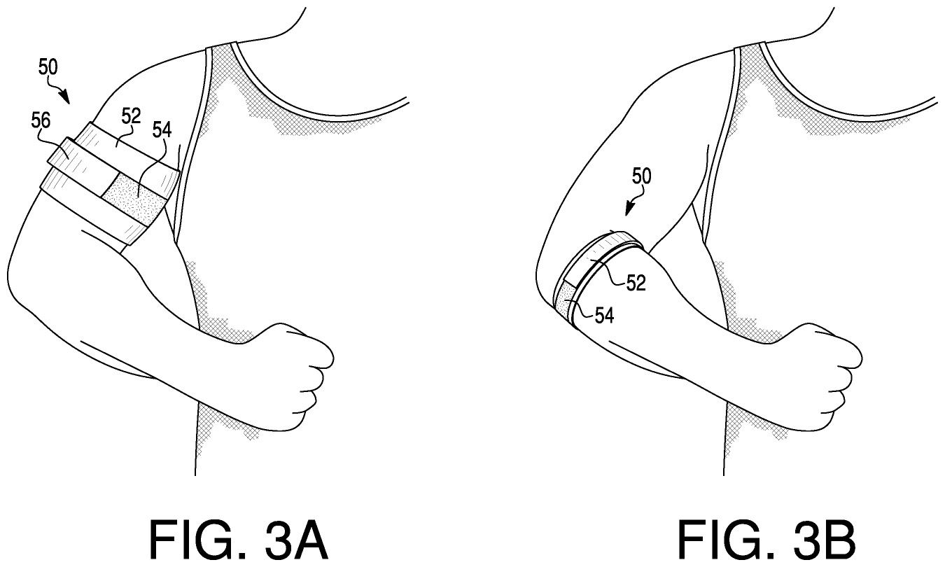

FIGS. 3A-B depict another embodiment of an exemplar modular wearable device 50 being utilized in various methods. The wearable device 50 can be worn as an armband, as shown in FIG. 3A, or positioned on the neck, the chest, or on the forearm or the wrist as a bracelet/watch, as shown in FIG. 3B. In all of these cases the wearable device 50 can be positioned as an accessory or integrated into an apparel piece residing on the user. The wearable device 50 includes a cuff 52 or portion that wraps around a user's arm, and the cuff engages with hook-and-loop fasteners 54, or a similar fastener, to stay in place on the user's arm. The physiological sensors may be integrated within the cuff 52 or may be centrally located in a centralized component 56, as shown in FIG. 3A, which may be detachable from the cuff 52 for washing of the cuff. The wearable device 50 is preferably battery operated with rechargeable batteries that may be charged on any typical charging pad or plugged into household electricity via a charging cord, such as with a USB cable or similar, as with any conventional mobile device.

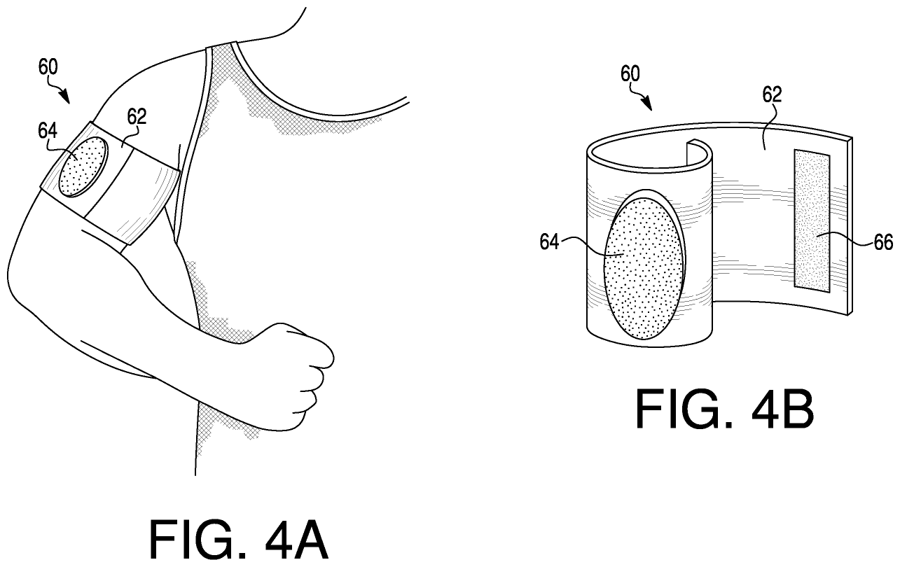

FIGS. 4A-B depict yet another embodiment of an exemplar modular wearable device 60 being utilized in various methods. The wearable device 60 is capable of being detached and utilized as an accessory or integrated into apparel. The wearable device 60 includes a spring-loaded cuff portion 62, having the ability to wrap around surfaces such as an arm or pole. The wearable device 60 can serve as an armband, an attachment to a pole or other inanimate objects, it can be connected as a broch, or worn as a watch. The attachment methods may include a hook and loop fasteners, such as VELCRO® brand fasteners shown at reference numeral 66, clasping system, elastic straps, adhesives, clip in/on connectors. The physiological sensors may be integrated within the cuff 62 or may be centrally located in a centralized component 64, which may be detachable from the cuff 52 for washing of the cuff. The wearable device 60 is preferably battery operated with rechargeable batteries that may be charged on any typical charging pad or plugged into household electricity via a charging cord, such as with a USB cable or similar, as with any conventional mobile device.

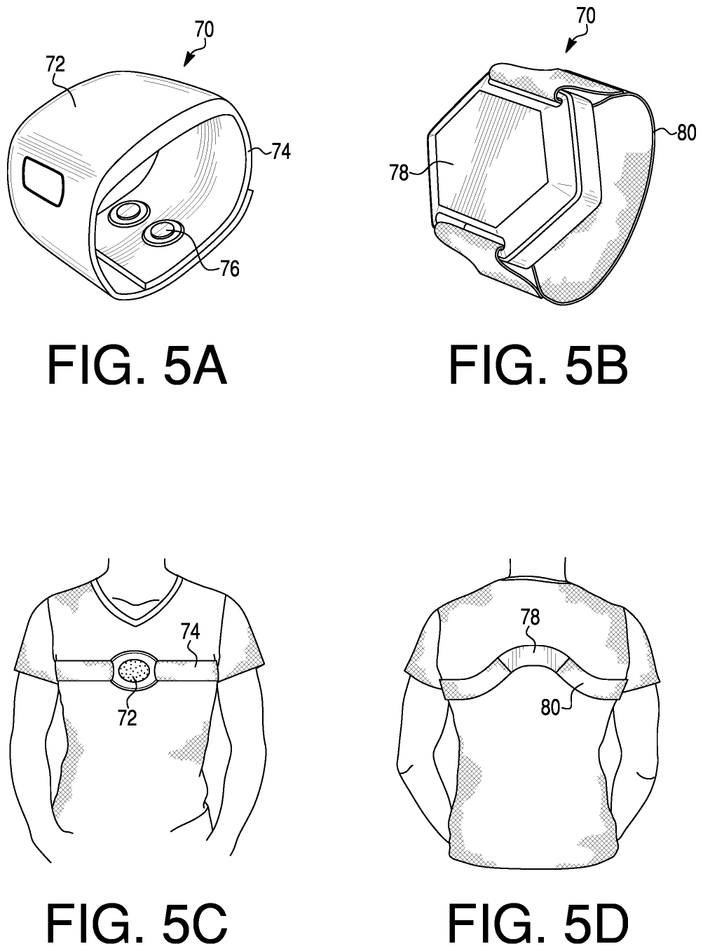

FIGS. 5A-D depict still another embodiment of an exemplar modular wearable device 70 being utilized in various methods. The wearable device 70 includes a centralized component 72, which may include physiological sensors, such as ECG and EMG sensors. The wearable device 70 includes a band 74 with clasps 76, as shown in FIG. 5A, which allows a user to wear in a fashion similar to a watch. Alternatively, the band may be of an elastic material, such as that shown at reference numeral 80 in FIG. 5B, and sufficiently large so as to wear about a person's chest, as shown in FIGS. 5C and 5D. A second centralized component 78 may be positioned along a person's back or spine to monitor for neuromuscular issues. The wearable device 70 is preferably battery operated with rechargeable batteries that may be charged on any typical charging pad or plugged into household electricity via a charging cord, such as with a USB cable or similar, as with any conventional mobile device.

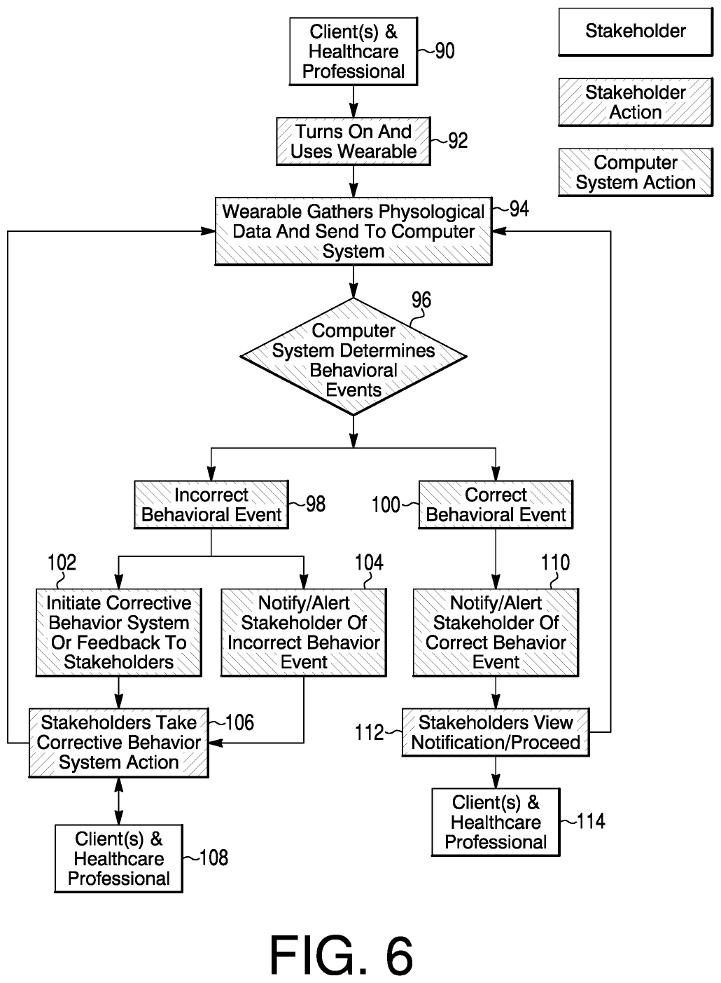

FIG. 6 depicts a high-level view of the functionality of the present invention for determination of behavioral events and subsequent actions. The diagram shows how a wearable device is utilized to gather physiological data and send it to the computer system. The computer system platform then determines behavioral events and takes action. In the case of the correct behavior event occurring then the stakeholder is notified and either takes action or proceeds with the information. In the case that the incorrect behavioral event occurs, then the system initiates the corrective behavior system, by notifying the stakeholder of incorrect behavior then providing a form of haptic, visual, audio, and/or any other combination of feedback to stakeholders. The stakeholder then undergoes the corrective behavior system action and mitigates or adjusts the incorrect behavior. In either case the system repeats the cycle of gathering physiological data and making determinations.

Specifically, as shown in FIG. 6, the process begins with a consulation between a healthcare professional and a patient (reference numeral 90) in which certain behavioral events are identified for correction. The patient wears a wearable device (reference numeral 92), such as one of the several embodiments disclosed herein, depending on the patient's condition and the behavior sought to be corrected. The wearable device gathers physiological data and sends to a computer system (reference numeral 94). During the course of a typical day-to-day in the life of the patient, the wearable device, in communication with the computer system, monitors certain physiological data to determine behavioral events (reference numeral 96). If incorrect behavioral events are determined (reference numeral 98), then the computer system notifies the patient and/or the healthcare professional of the behavioral event (reference numeral 104) while at the same time initiating corrective behavioral feedback (reference numeral 102), such as a predetermined haptic effect to promote corrective behavior by the patient (reference numeral 106). The determination of incorrect behavioral events can be an iterative process between the healthcare professional and the patient as appropriate corrective action is assessed and implemented (reference numeral 108). Alternatively, if the wearable device and computer system determine that the patient is exhibiting the correct behavioral event (reference numeral 100), reward notifications may be generated (reference numeral 110), encouraging the patient to continue the correct behavior (reference numeral 112), and these notifications may be provided to the healthcare professional or automatically uploaded into the patient's charts or medical files for reference by the healthcare provider (reference numeral 114).

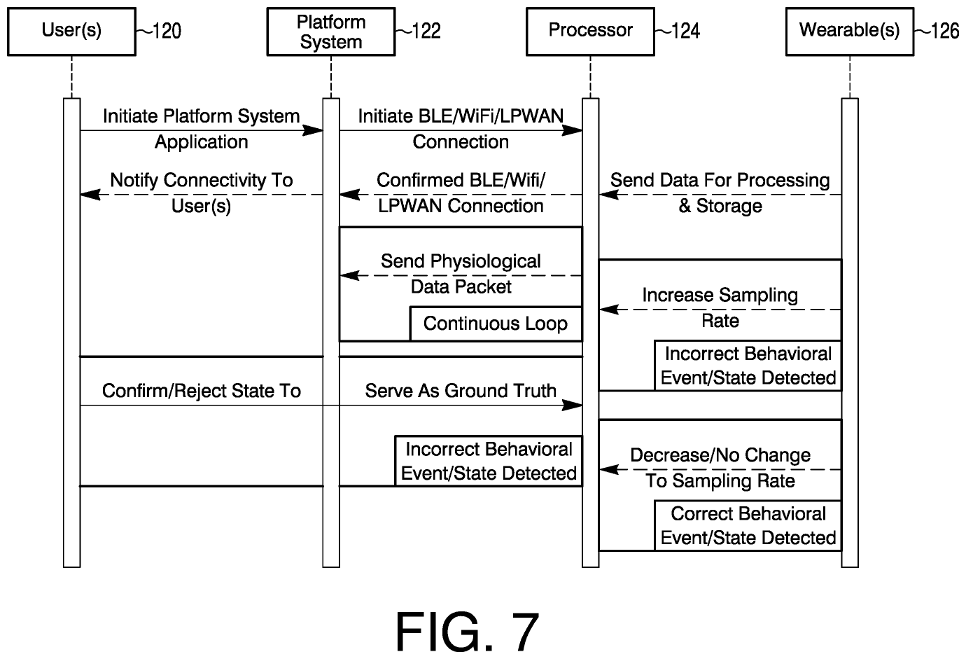

FIG. 7 depicts the data flow analyzed by the present invention between the user(s) 120, computer system 122, processor unit 124, and wearable device(s) 126 as it relates to the incorrect behavior being the desired outcome. The computer system 122 may include mobile compatible web applications, native mobile and/or computer/desktop applications, web applications, third-party plug-ins, third-party smart devices (i.e. Apple Watch, Garmin, Fitbit, etc.), primary party smart devices (i.e. custom made), among other devices.

As shown in FIG. 7, the user(s) 120 initiates the computer system 122, enabling the wireless connection between the computer system 122 and the processor 124 affixed in the wearable device 126. Once enabled, the processor 124 confirms connection to the computer system 122 through a series of secured wireless data “handshakes,” using secure protocols such as SSL or TLS. Once the connection is established, real time connection physiological data and behavioral events are reported to the user 120. The processor 124 sends data packets, consisting of compressed physiological fused data, to the computer system 122 at a consistent static time interval. Once either the user 120 or the platform system 122 confirms or rejects a behavior event, the processor 124 begins sending data packets at an increased interval, to the computer system 122, which relays the data to a server connected to the computer system 122. The increased rate of data transmission allows for higher fidelity readings during a detected behavior event.

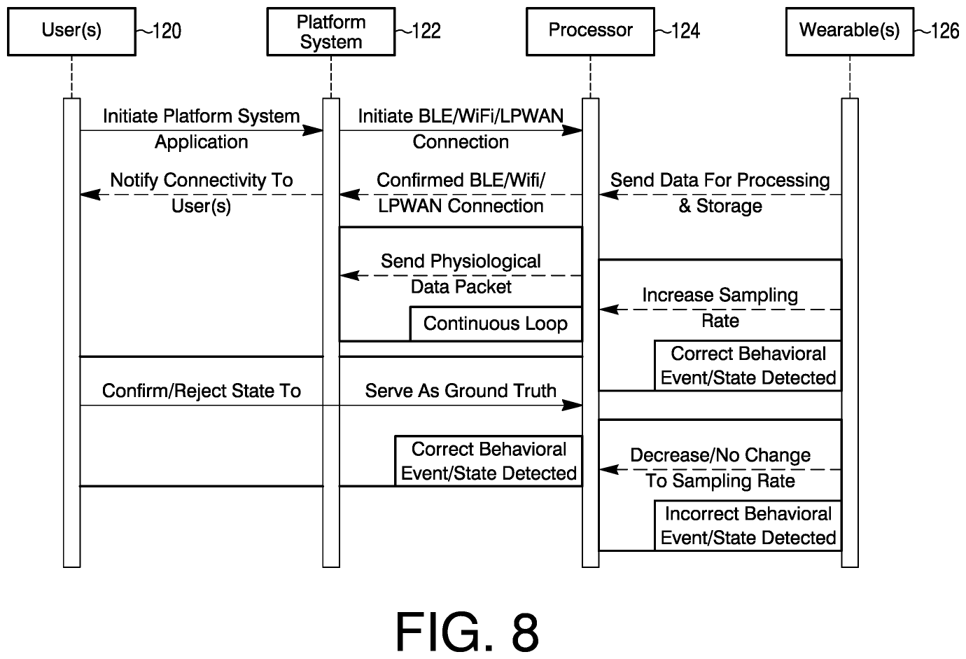

FIG. 8 depicts the data flow analyzed by the present invention between the user(s) 120, computer system 122, processor unit 124, and wearable device(s) 126 as it relates to the correct behavior being the desired outcome. The user(s) 120 initiates the computer system 122, enabling the wireless connection between the computer system 120 and the processor 124 affixed in the wearable device 126. Once enabled, the processor 124 confirms connection to the computer system 122 through a series of secured wireless data “handshakes.” Once the connection is established, real time connection physiological data and behavioral events are reported to the user 120. The processor 124 sends data packets, consisting of compressed physiological fused data, to the computer system 122 at a consistent static time interval. Once either the user 120 or the platform system 122 confirms or rejects a behavior event, the processor begins sending data packets at an increased interval, to the computer system, which relays the data to a server. The increased rate of data transmission allows for higher fidelity readings during a detected behavior event.

The present invention has the capability to provide user-friendly visualizations of therapeutic outcomes, carry-over, and independence of client/patients/healthcare professionals to prompt better engagement and care. The system has capabilities of providing proactive care solutions of incorrect/undesired behaviors to not only allow for better future/long-term planning but also immediate/short-term care and action for the given stakeholders.

The wearable device(s) (for instance, reference numerals 40, 50, 60, or 70) utilize an array of inputs to gather, determine, and assess from sensors that monitor physiological data related to cardiovascular, endocrine, neurological, cognitive, emotional, physical orientation and positioning, force and power, among others. The blend of physiological data that is collected is able to extract the behavioral event.

An example of short-term care action includes but not limited to health care professionals being alerted that the client, an individual with anxiety disorders or Autism Spectrum Disorder (ASD), is at high risk of undesired behaviors and provided information to assist in mitigation. An example of long-term care is the client is able to visually see how their engagement in and out of healthcare professional sessions tied with the physiological data has led them to their independence. The solution will be able to provide early notifications of behavioral events to user(s), allowing them to proactively take corrective actions in addressing the correct or incorrect behavioral events.

By conveying this information to the user(s) via computer systems with instructions for corrective actions, the solution enables stakeholders and user(s) with a plethora of varying degrees backgrounds to have better data-driven insights.

For instance those with and their communities of cognitive & emotional disorders as well as physiological & mental disabilities can benefit by having a platform to improve transparency, communication, objective data-driven insights, and more “user-friendly” insights to present correlation between engagement to behavioral regulation. This can then lead to those individuals to regain independence from caregivers and conventional means of handling independence inhibiting behavioral events.

In another instance skeletal muscle, muscle detection, body position sensors allow for real time assessment of muscular rehabilitation, prehabilitation, and injury prediction. This data is stored in a server, allowing access to third parties including, but not limited to, doctors, therapists, insurance companies, caregivers, and guardians to better assess and treat potential injuries of individuals.

In another example, consider an individual who may be at high risk of or recovering from a muscle injury. Muscle damage can be in the form of tearing (part or all) of the muscle fibers and the tendons attached to the muscle. The leading causes of muscle strain or muscle tears are overloading of muscles, fatigue-related muscle strain, and an overdose of exercise. Electromyography and body position may be used to obtain benchmark measurement signals to detect if an individual is overloading their muscles. Also, electromyography can be used to compare a muscle signal to the benchmark signal during rehabilitation. Multiple electromyography and body position signals may be used to measure muscle imbalances to prevent fatigue-related muscle strain. According to principles of this invention, muscle strain can be automatically detected through the use of wearable physiological sensors to detect electromyography activity, and send a notification to the individual or third party.

In another example, consider a person with Epilepsy, who may encounter a loss of consciousness and muscle contraction that results in a seizure. During a muscle contraction, muscle fibers emit an electrochemical signal that may be detected through electromyography. Electromyography may be used to obtain benchmark measurement signals to detect if an epileptic individual is experiencing severe muscle contractions. According to principles of this invention, muscle contraction can be automatically detected through the use of wearable physiological sensors to detect electromyography activity and send a notification to a third party.

In another example, consider an individual recovering from a stroke, who may get frustrated with the slow rehabilitation process. According to principles of this invention, muscle contraction can be automatically detected through the use of wearable physiological sensors to detect electromyography activity and send a notification to a third party.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended set of claims.

What is claimed is:

1. A platform system for remotely monitoring one or more behavioral event of at least one user, the platform system comprising:
at least one computer system having an internet connection and at least one web application, said at least one computer system having a memory for storing an array of contextual data relating to said at least one user;
at least one wearable device having a processor and being communicatively connected to said at least one user and being in electronic communication with said at least one computer system, said at least one wearable device having one or more sensors for detecting physiological data of said at least one user, said at least one wearable device having a memory for storing an array of physiological data detected by said one or more sensors;
wherein said processor of said at least one wearable device determines a behavioral event relating to said at least one user based on said array of physiological data in view of said array of contextual data, said processor communicating said behavioral event to said computer system; and
wherein said computer system provides a notification relating to said behavioral event.

2. The platform system of claim 1 wherein the computer system is selected from the group consisting of mobile compatible web applications, native mobile and/or computer/desktop applications, web applications, third-party plug-ins, third-party smart devices, and primary party smart devices.

3. The platform system of claim 1 wherein the array of physiological data is selected from the group consisting of Electrocardiography (ECG), Heart Rate (HR), Heart Rate Variability (HRV), Photoplethysmography (PPG), Pulse-Rate-Variability (PRV), Inter-Beat-Interval (IBI), Electrodermal Activity (EDA), Body Temperature, Respiratory Rate (RR), Blood Pressure (BP), Electromyography (EMG), Peripheral Capillary Oxygen Saturation (Sp02), Peripheral Capillary Deoxygenation (SpD02), Inertial Measurement Unit (IMU), Global Positioning System (GPS), Speed (distance per time), Acceleration (distance per time squared), and Force (Newtons).

4. The platform system of claim 1 wherein the array of contextual data is selected from the group consisting of user-inputted information, personal demographic & health information, calendar events, Rated Perceived Exertion (RPE), Emotional Perceived Exertion (EPE), dietary, exercise/exercise completion, assessments of professionals/health care professionals/care providers/therapist, environmental conditions, geographical location, altitude, temperature, humidity, and audio.

5. The platform system of claim 1 wherein said behavioral event is selected from the group consisting of behavioral dysregulation, anxiety attacks, autism spectrum disorder (ASD), strokes, seizures, labor contractions/when to push during labor, physical form detection, force production, Sudden infant death syndrome (SIDS), neurological disorders, dysregulation, physical therapy, eating disorders, smoking, post traumatic stress disorder (PTSD), and gaming.

6. The platform system of claim 1 wherein said computer system further provides a corrective action to said at least one user upon detection of said behavioral event.

7. The platform system of claim 6 wherein the corrective action is selected from the group consisting of alleviation, resolutive, or reminder action of the correct or incorrect behavior may be pulsation (making the heart tempo to reduce beats per minute), comprised of compression of user, visually, audibly/haptic reminder to user of what to do in accordance to prior procedure as indicated by health care professionals and other caregivers, visual, written, and auditory feedback.

8. The platform system of claim 6 wherein the corrective action is used for alleviation and/or self-correction of intended addressable behaviors.

9. A platform system for increasing patient carry-out for in and out of a patient's therapy exercise, the platform system comprising:
at least one computer system having an internet connection and at least one web application, said at least one computer system having a memory for storing an array of contextual data relating to said patient;
at least one wearable device having a processor and being communicatively connected to said patient and being in electronic communication with said at least one computer system, said at least one wearable device having one or more sensors for detecting physiological data of said patient, said at least one wearable device having a memory for storing an array of physiological data detected by said one or more sensors;
wherein said processor of said at least one wearable device determines completion of a portion of said therapy exercise relating to said patient based on said array of physiological data in view of said array of contextual data, said processor communicating said completion to said computer system; and
wherein said at least one wearable device provides said patient with feedback to motivate completion of said therapy exercise.

10. The platform system of claim 9 wherein the feedback is selected from the group consisting of auditory response, haptic response, and visual response.

11. The platform system of claim 9 wherein the computer system is selected from the group consisting of mobile compatible web applications, native mobile and/or computer/desktop applications, web applications, third-party plug-ins, third-party smart devices, and primary party smart devices.

12. The platform system of claim 9 wherein the array of physiological data is selected from the group consisting of Electrocardiography (ECG), Heart Rate (HR), Heart Rate Variability (HRV), Photoplethysmography (PPG), Pulse-Rate-Variability (PRV), Inter-Beat-Interval (IBI), Electrodermal Activity (EDA), Body Temperature, Respiratory Rate (RR), Blood Pressure (BP), Electromyography (EMG), Peripheral Capillary Oxygen Saturation (Sp02), Peripheral Capillary Deoxygenation (SpD02), Inertial Measurement Unit (IMU), Global Positioning System (GPS), Speed (distance per time), Acceleration (distance per time squared), and Force (Newtons).

13. The platform system of claim 9 wherein the array of contextual data is selected from the group consisting of user-inputted information, personal demographic & health information, calendar events, Rated Perceived Exertion (RPE), Emotional Perceived Exertion (EPE), dietary, exercise/exercise completion, assessments of professionals/health care professionals/care providers/therapist, environmental conditions, geographical location, altitude, temperature, humidity, and audio.

14. The platform system of claim 9 wherein the carry-over therapy exercise is selected from the group consisting of engagement or compliance with prescribed activities, coping mechanisms, and participation in interactions with the computer system with wearable technology.

15. A platform system for remotely monitoring one or more behavioral event of at least one user, the platform system comprising:
at least one computer system having an internet connection and at least one web application, said at least one computer system having a memory for storing an array of contextual data relating to said at least one user;
at least one wearable device being communicatively connected to said at least one user and being in electronic communication with said at least one computer system, said at least one wearable device having a central processing unit operatively connected to an array of sensors for detecting physiological data of said at least one user, said at least one wearable device having a memory for storing an array of physiological data detected by said array of sensors;
wherein said central processing unit of said at least one wearable device determines a behavioral event relating to said at least one user based on said array of physiological data in view of said array of contextual data, said processor communicating said behavioral event to said computer system; and
wherein said central processing unit provides corrective behavior to said at least one user after detecting said behavioral event.

16. The platform system of claim 15 wherein the detected behavioral event is selected from the group consisting of emotional, physiological, musculoskeletal, neuromusculoskeletal, mental, and contextual experiences affecting said at least one user.

17. The platform system of claim 15 wherein said behavioral event is selected from the group consisting of behavioral dysregulation, anxiety attacks, autism spectrum disorder (ASD), strokes, seizures, labor contractions/when to push during labor, physical form detection, force production, Sudden infant death syndrome (SIDS), neurological disorders, dysregulation, physical therapy, eating disorders, smoking, post traumatic stress disorder (PTSD), and gaming.

18. The platform system of claim 15 wherein the at least one wearable device is capable of being worn on the wrist, forearm, bicep, foot, ankle, leg, chest, head, neck, or pelvic region.

19. The platform system of claim 15 wherein the at least one wearable device is configured to be worn with an article of clothing.

20. The platform system of claim 15 wherein the at least one wearable device is configured to be worn as an accessory to the at least one user's body, wherein the array of sensors is communicatively connected to said at least one user's body.
